(19) United States
(12) Patent Application Publication
(10) Pub. No.: US 2005/0228252 A1
(43) Pub. Date: Oct. 13, 2005

(54) Electrophysiology system and method

(75) Inventor: Curtis G. Neason, New York, NY (US)

Correspondence Address: GE MEDICAL SYSTEM;C/O FOLEY & LARDNER, 777 EAST WISCONSIN AVENUE, MILWAUKEE, WI, 53202-5367, US

(73) Assignee: General Electric Company

(21) Appl. No.: 10/817,143

(22) Filed: Apr. 2, 2004

(51) Int. Cl. A61B 5/05

(52) U.S. Cl. 600/407

(57) ABSTRACT

The subject matter described herein pertains to a system that comprises one or more probes configured to be positioned inside a heart of a patient, a processor communicatively coupled to the one or more probes, a display communicatively coupled to the processor, and image processing tools which are used by the processor to manipulate the image. The processor being used to process electrical information pertaining to the heart. The electrical information is sensed using the one or more probes. The display is used to display an image of the heart.

### BACKGROUND

EP studies can be used to diagnose and treat a number of serious heart problems. One type of heart problem that can be diagnosed and treated by conducting an EP study is a cardiac arrhythmia. A cardiac arrhythmia can generally be referred to as an abnormal heart rhythm such as tachycardia, bradycardia, etc. One particularly dangerous arrhythmia that is often diagnosed and treated using an EP study is ventricular fibrillation. Left untreated, an arrhythmia presents a serious health risk to an individual.

Various types of systems may be used to perform various procedures in an EP study such as an EP mapping procedure or an EP monitoring and diagnostic procedure. For example, an EP mapping system is used to perform an EP mapping procedure. In an EP mapping procedure, a catheter is inserted into a vein or artery (e.g., in the groin, etc.) and guided to the interior of the heart. Once inside the heart, the catheter is contacted with the endocardium at multiple locations. At each location, the position of the catheter and the activation time can be measured. This information may then be used to create a structural map of the heart showing the activation times. The attending physician uses this information to assist in locating the origin of a cardiac arrhythmia. Generally, once the origin of the arrhythmia is located, the area is ablated using the catheter.

In other instances, an EP monitoring system may be used to acquire more information pertaining to the heart. For example, EP monitoring systems may be used to pace the heart and record the resulting electrical activity. In some EP studies, the physician performing the study may refer to an image of the heart to, among other things, ascertain the unique anatomical features of each patient's heart. Typically, the images are taken in the radiology department before the EP study begins. For example, images that may be used include computed tomography images (CT), magnetic resonance images (MR), positron emission tomography images (PET), etc. Once the image has been acquired, the image may be saved on a network.

Previously, a dedicated workstation has been provided in the EP lab to retrieve the images off the network and further manipulate them as desired. The workstation that is used to retrieve, store, and manipulate the images is separate from the EP monitoring and/or mapping system which is used to perform this EP study. Thus, two computer systems are often used in the EP lab to perform the EP procedure. Accordingly, it would be desirable to provide an improved EP monitoring and/or mapping system that was capable of retrieving, storing, and/or manipulating the images. Also, including the images with the EP systems provides additional advantages such as being able to prepare a report that includes the image, use image processing tools to provide additional views of the image as the procedure progresses, etc. Unfortunately, including image processing tools on an EP system is no simple task since the EP monitoring and/or mapping system is considered a medical device and is subject to strict regulations and requirements imposed by the various governments where they are used. Also, combining image processing tools with EP systems is typically difficult because an EP system uses different software tool sets than an image review system because of the differing nature of the data included in each system (e.g., EP systems are generally configured to record and manipulate waveforms, etc. rather than images). Moreover, both systems are typically coupled to separate networks (e.g., many hospitals and other medical complexes may use separate networks to store EP data and images) and the systems may use different hardware (e.g., an image review system or station may use a high-end video board for good image quality whereas the EP system may use a high-end CPU for real-time data acquisition).

The claims define the scope of the subject matter for which protection is sought, regardless of whether any of the aforementioned disadvantages are overcome by the subject matter recited in the claims. Also, the terms recited in the claims should be given their ordinary and customary meaning as would be recognized by those of skill in the art, except, to the extent a term is used herein in a manner more expansive than its ordinary and customary meaning, the term should be given its ordinary and customary meaning plus the additional expansive meaning, or except if a term has been explicitly defined to have a different meaning by reciting the term followed by the phase “as used herein shall mean” or similar language. Accordingly, the claims are not tied to any particular embodiment, feature, or combination of features other than those explicitly recited in the claims. This is true even in situations where only one embodiment is described herein but the claims use one or more terms having an ordinary meaning that would encompass other embodiments not specifically referenced herein.

### SUMMARY

According to one embodiment, a system comprises one or more probes configured to be positioned inside a heart of a patient, a processor communicatively coupled to the one or more probes, a display communicatively coupled to the processor, and image processing tools which are used by the processor to manipulate the image. The processor being used to process electrical information pertaining to the heart. The electrical information is sensed using the one or more probes. The display is used to display an image of the heart.

According to another embodiment, a computer based system comprises electrophysiology monitoring logic which is used to monitor and control one or more probes positioned inside a heart, mapping logic which is used create a structural map of the heart by determining the position of at least one of the one or more probes inside the heart, and image processing logic which is used to manipulate an image of the heart. The one or more probes is used to sense electrical information pertaining to the heart.

According to another embodiment, a system comprises one or more probes configured to be positioned inside a heart, a data processing system communicatively coupled together and communicatively coupled to the one or more probes,. At least one of the one or more probes is used to sense electrical information pertaining to the heart. The data processing system is configured to store position information pertaining to a position of at least one of the one or more probes. The data processing system is also configured to store an image of the heart and image processing tools. The image processing tools are used to manipulate the image.

According to another embodiment, a combination system comprises an electrophysiology monitoring system which is configured to be communicatively coupled to one or more probes positioned inside a heart, an electrophysiology three-dimensional mapping system which is configured to receive position information pertaining to the position of the one or more probes, and image processing logic which is used to manipulate an image of the heart. The one or more probes being configured to sense electrical information pertaining to the heart. The position information is used to create a three-dimensional structural map of the heart. The electrophysiology monitoring system and the electrophysiology three-dimensional mapping system are communicatively coupled together.

According to another embodiment, a system comprises one or more probes configured to be positioned inside a heart of a patient, a processor communicatively coupled to the one or more probes, image processing tools in the form of computer readable instructions, and a user interface communicatively coupled to the processor. The processor is used to process electrical information pertaining to the heart. The electrical information is sensed using the one or more probes. The image processing tools are used to manipulate an image of a heart. The image is constructed based on a plurality of image slices each of which represents a cross sectional slice of the heart. The user interface is configured to display the image.

### BRIEF DESCRIPTION OF THE DRAWINGS

FIG. 1 is an electrophysiology system used to monitor and/or map a heart according to one embodiment.

FIG. 2 shows a network which includes the system of FIG. 1 according to another embodiment.

FIG. 3 is a flowchart showing one embodiment of a method for receiving and/or storing images of a heart according to another embodiment.

FIG. 4 is an electrophysiology system used to monitor and/or map a heart according to another embodiment.

### DETAILED DESCRIPTION

The subject matter described herein is generally referred to in the context of a system which is configured to receive and/or store information pertaining to the heart of a patient (e.g., electrical information pertaining to the heart, structural information pertaining to the heart, images of the heart, etc.). Although, the present description is provided primarily in the context of receiving, storing, and using electrical information, structural information, and/or images, it should be understood that the systems and methods described and claimed herein may also be used in other contexts as would be recognized by those of ordinary skill. It should also be understood that a particular example or embodiment described herein may be combined with one or more other examples or embodiments also described herein to form various additional embodiments as would be recognized by those of ordinary skill. Also, reference to various features in singular tense should also be understood as equally including the plural tense unless noted otherwise. Accordingly, the systems and methods described herein may encompass various embodiments and permutations as may be desired.

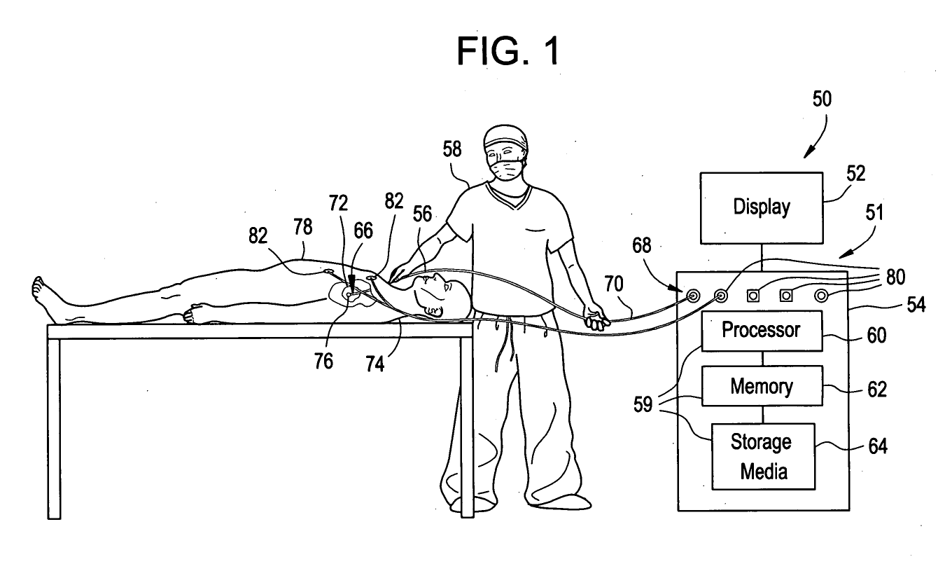

Referring to FIG. 1, one embodiment of an electrophysiology system 50 (e.g., and EP monitoring system, EP mapping system, etc) is shown. System 50 includes a console or computer 51 and a probe 56. System 50, broadly described, may be used to receive, store, and display various types of information. In particular, system 50 may be used to simultaneously and/or selectively receive, store, and/or display electrical information, structural information, and/or images pertaining to a heart 72 of a patient 74.

In general, system 50 may be any system that is configured to use one or more probes 56 positioned inside the body to measure, monitor, diagnose, manipulate, and/or otherwise provide information about heart 72. In particular, system 50 may be an EP mapping system. The EP mapping system may generally be used to store position information pertaining to the position of probe 56. The position information may be used to create a structural map of heart 72. In another embodiment, system 50 may be an EP monitoring system. Unlike an EP mapping system, an EP monitoring system is not used to create a structural map of the heart. Rather, the EP monitoring system is used to perform additional and more detailed electrical analysis and stimulation of heart 72. For example, the EP monitoring system may be used to pace heart 72 and record or log electrical information while the heart is being paced. In another embodiment, system 50 may be a combination of both an EP mapping system and an EP monitoring system.

As shown in FIG. 1, probe 56 and a display or user interface 52 are communicatively coupled to a data processing system 54, which includes data processing components 59. Information sensed by probe 56 may be communicated to data processing system 54. Information from data processing system 54 may then be communicated to display 52 where it is displayed to a nearby person 58 (e.g., attending physician, nurse, technician, etc.). The configuration shown in FIG. 1 is only one of many suitable configurations. For example, in another embodiment, probe 56 may be communicatively coupled directly to display 52. In this embodiment, display 52 may be combined with data processing system 54 so that the functions generally performed by data processing system 54 and display 52 are performed by the combined unit (e.g., display 52 comprises all of data processing components 59). In another embodiment, console 51 may include two or more displays 52. For example, one display may be used to display electrical and/or structural information pertaining to heart 72 and the other may be used to display an image such as a computed tomography (CT), magnetic resonance MR, ultrasound, and/or positron emission tomography (PET) image. Of course, a wide variety of information may be displayed on display 52. In one embodiment, display 52 may be configured to be at a position that is convenient for person 58 to view (e.g., display 52 is positioned at eye level of person 58 when person 58 is standing, etc.) as person 58 moves probe 56.

System 50 may be configured to include additional components and systems. For example, system 50 may comprise a printer. The printer may be configured to print on standard sized paper or may be configured to print on smaller rolls of paper. The printer may also be used to print out a report at the end of an EP study.

In one embodiment, system 50 is configured to allow a user to enter notes about the patient/procedure. Also, system 50 may be configured to log the details of a particular EP study (e.g., medication information such as type, amount, times administered, etc., pacing information, ablations information, etc.). This data is typically logged at regular intervals throughout the procedure. This data can be used during the procedure or after the procedure to further analyze and diagnose the problem. This may be useful when the problem continues even after the procedure is over.

In another embodiment, system 50 may be configured to include a number of individual commands. These individual commands may be combined in a single command using manufacturer configured or user configured macros. The macros allow the user to customize system 50 in any desirable manner. For example, a macro may be created which is used to end a case. When this macro is activated, system 50 is commanded to take a final vitals measurement, print a report, and stop recording electrical information. Thus, the user does not need to perform each of these commands separately each time a procedure is completed.

In another embodiment, system 50 may be configured to use a Holter window. Also, system 50 may be configured to include an alignment window, which allows the user to compare signals from different locations to each other.

System 50 may be configured to receive, store, and/or display various information pertaining to patient 74. For example, in one embodiment, system 50 may be configured to receive, store, and/or display vitals information pertaining to patient 74. Vitals information may include one or more, in any combination, of the following types of patient information: pulse oximetry (SpO₂, non-invasive blood pressure (NIBP), temperature, respiratory rate, respiratory CO₂ concentration (etCO₂ ), impedance cardiography (ICG), pulse rate, cardiac output (CO), etc. System 50 may also include sensors that are communicatively coupled to data processing system 54 in console 51 to provide this information. In one embodiment, display 52 may be configured to display at least one, two, three, four, or all five of the following types of information pertaining to patient 74: non-invasive blood pressure, temperature, respiratory rate, pulse oximetry, respiratory CO₂ concentration, and pulse rate.

Data processing system 54, shown in FIG. 1, comprises data processing components 59, which includes a processor 60, memory 62, storage media 64, and one or more input devices (e.g., mouse, keyboard, etc.). Data processing system 54 is configured to receive information from probe 56, process the information, and provide output using display 52. The information provided to data processing system 54 may be continually stored (i.e., all information is stored as it is received) or intermittently stored (i.e., periodic samples of the information are stored or logged) using storage media 64 (e.g., optical storage disk such as a CD, DVD, etc., high performance magneto optical disk, magnetic disk, etc.). In general, storage media 64 differs from memory 62 in that storage media 64 is configured to maintain the information even when storage media 64 is not provided with power. In contrast, memory 62 typically does not maintain the information when the power is off.

In one embodiment, console 51 is a desktop computer. In another embodiment, console 51 may include input receivers 80 that are configured to receive additional information pertaining to patient 74. For example, in one embodiment, input receivers 80 may include one or more input receivers configured to receive input from leads 82 (e.g., ECG leads, etc.). In other embodiments, input receivers 80 may include suitable receivers for receiving vitals information. For example, input receivers 80 may be configured to be coupled to a traditional NIBP arm cuff sensor.

Probe 56 comprises a distal end 66, a proximal end 68, and a probe body 70. In general, probe 56 may be positioned in or adjacent to heart 72 (shown in FIG. 1 in a cross-sectional view to expose distal end 66 of probe 56) of patient 74. In one embodiment, distal end 66 may include one or more sensors 76, which are configured to sense various electrical information (e.g., electrical potential at one or more positions of the endocardium, activation times, etc.) pertaining to heart 72. The electrical information may then be communicated back to console 51 and displayed on display 52 or stored on storage media 64. In one embodiment, probe 56 may comprise a plurality of sensors configured to sense the electrical information pertaining to heart 72 (e.g., probe 56 is a balloon or sock catheter, etc.). The electrical information may be used to create an electrical map (e.g., map of the activation times, electrical potentials, etc.) of heart 72.

Probe 56 may be any number of suitable probes having a variety of configurations. For example, probe 56 may include a lumen in which wires may be placed to communicate information from sensors 76 back to console 51 and to transmit an ablation charge from console 51 to distal end 66 to correct the electrical pathways in heart 72. Of course, the lumen may also be used to allow fluid to flow through probe 56.

In another embodiment, a localization system, included as part of system 50, may be used to determine the spatial location of one or more portions (e.g., sensors 76, etc.) of distal end 66 of probe 56. This may be useful in moving probe 56 back to an earlier position or to create a structural map of heart 72. Any suitable localization system may be used as would be recognized by those of ordinary skill. For example, the position of distal end 66 of probe 56 may be determined using one or more transmitters and/or receivers that are located outside the body of patient 74 (typically at least three transmitters and/or receivers are used). In this example, the transmitters and/or receivers may be configured to send and/or receive signals to and/or from distal end 66. These signals may be used to determine the position of distal end 66. In one embodiment, the transmitters and/or receivers may be incorporated into one or more leads 82 positioned on skin surface 78 of patient 74. In another embodiment, the transmitters and/or receivers may be positioned so as not to be in contact with patient 74. In another embodiment, leads 82 may be used to determine the position of distal end 66 of probe 56 by sending a signal that is useful in determining the impedance of probe 56, which may be used to determine the position of probe 56. In another embodiment, the localization-system may be configured to determine the position of multiple sensors 76 on distal end 66 of probe 56. In another embodiment, the position of probe 56 may be determined using a magnetic field.

Display 52, shown in FIG. 1, is configured to provide output to a user such as alphanumeric (e.g., text, numbers, etc.) output, graphical image output, etc. In one embodiment, display 52 may be configured to also receive input from a user (e.g., touch screen, buttons located adjacent to the screen portion of display 52, etc.). Display 52 may be any number of suitable displays in any number of suitable configurations. For example, display 52 may be a liquid crystal display, flat screen display, SVGA display, VGA display, etc.

In one embodiment, display 52 may be configured to display one or more images (CT, MR, ultrasound, PET, etc.) of heart 72. Display 52 may also be configured to display a structural and/or electrical map of heart 72. In another embodiment, display 52 may be configured to display vitals information pertaining to patient 74.

Display 52 may also be configured to display one or more representations of one or more probes 56 and the information provided by probes 56. For example, in one embodiment, display 52 may be configured to display a representation of probe 56. In another embodiment, display 52 may be configured to display representations of sensors 76 which are on probe 56. In another embodiment, display 52 may be configured to display the electrical information pertaining to heart 72, which is received from sensors 76 (e.g., a contour map of the electrical properties of heart 72). In another embodiment, display 52 may be configured to display markers showing one or more locations where the electrical information has been sensed. In one embodiment, each marker may display an abbreviated amount of information regarding the electrical information. When a user selects one of the markers, the user is shown a greater amount of electrical information for that particular location of heart 72. The markers may be color coded based on the activation times at the various locations inside heart 72 (e.g., red is for early activation times and blue is for late activation times). By displaying a number of markers on display 52, the user can readily observe the electrical information pertaining to various areas of heart 72. Any suitable marker or identifier may be used to represent probe 56 on display 52. For example, in one embodiment, probe 56 may be displayed as a line with a series of points corresponding to sensors 76. The line segments connecting the points represent the portion of probe 56 where there are no sensors. Probe 56 may be shown or represented on display 52 in any of a number of other suitable ways as well.

In one embodiment, system 50 may be configured to receive a real-time or live X-ray image (e.g., fluoroscopy image) and correlate the image to the electrical information sensed using probe 56. Typically, this is done by gating and correlating (e.g., temporally, reference tags, etc.) the image data and the electrical information as they are received by system 50. The image may be displayed alone or simultaneously (e.g., a live image and a stored image are displayed, etc). The images may be annotated with comments or to mark areas of interest (e.g., activation sites, etc.).

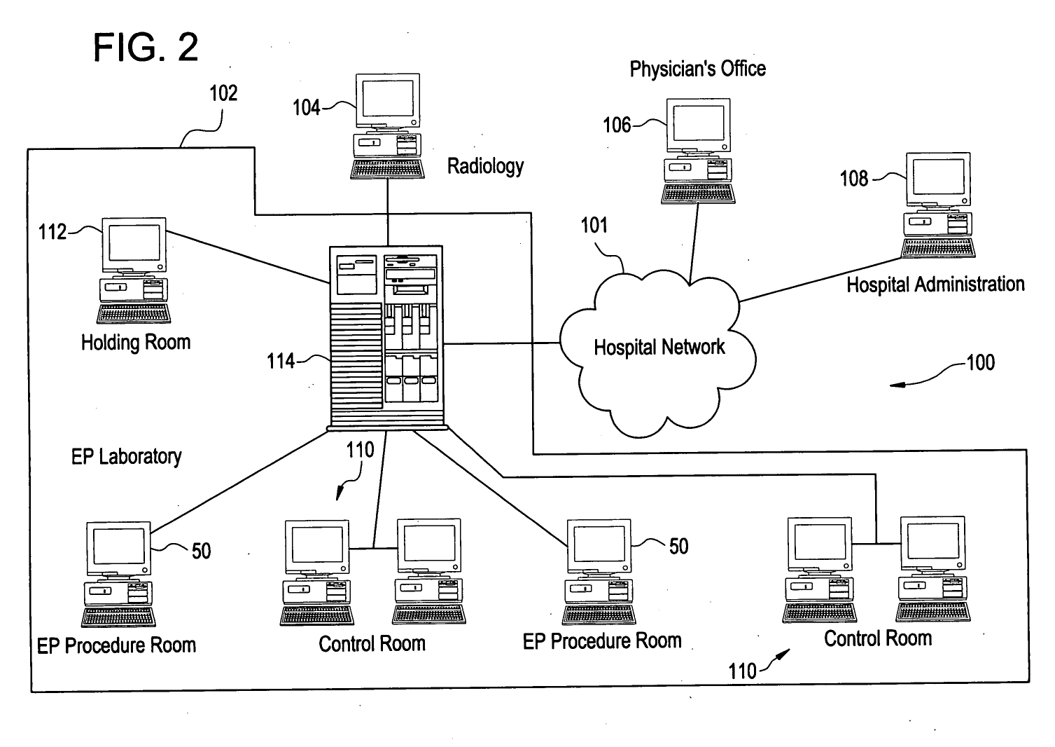

Referring to FIG. 2, system 50 may also be configured as part of a network 100 of computers (e.g., wireless, cabled, secure network, etc.). Network 100 may include any of a number of suitable computers and may be part of a number of suitable networks. For example, network 100 may be a health care facility network (e.g., hospital, clinic, etc.), the Internet, wide area network, etc. In the embodiment shown in FIG. 2, network 100 is a hospital network. Network 100 is used to transfer information between an EP laboratory 102, a workstation 104 located in a radiology laboratory, and other workstations 106, 108 located on hospital network 101.

As shown in FIG. 2, EP laboratory 102 comprises two EP procedure rooms where EP studies and procedures are performed with each EP procedure room including an adjacent control room with additional workstations 110, which are used to monitor the progress of an EP study. Also included as part of EP laboratory 102 is a holding room which includes a workstation 112. The holding room may be used as a place for patient 74 to wait immediately before and immediately after the EP procedure.

In one embodiment, file server or data storage system 114 is included as part of EP laboratory 102. File server 114 may include a database or other data storage and management system to store data from EP laboratory 102 or from other areas workstations on network 100. It should be understood that the configuration shown in FIG. 2, is only one example of network 100. In other embodiments, EP laboratory 102 may be configured to not include the holding room and associated workstation 112. Also, file server 114 may not be included as part of EP laboratory 102. Rather, file server 114 may be included as part of hospital network 101 that is outside of EP laboratory 102.

In the embodiment shown in FIG. 2, network 100 includes workstation 104 located in the radiology laboratory of the hospital. In one embodiment, workstation 104 may be an internal medical imaging system such as a CT, MR, ultrasound, PET, single-photon emission computed tomography (SPECT), optical coherence tomography (OCT) imaging system. Thus, the internal medical imaging system used to acquire images of heart 72 of patient 74 may be used to save the images directly to file server 114. In another embodiment, workstation 104 may be separate from the internal medical imaging system used to acquire an image of heart 72 of patient 74. Thus, the image data acquired using the imaging system may be saved to a portable data storage medium (e.g., writable CD, writable DVD, etc.), which may be used to transfer the image data to workstation 104. Once the image data is on workstation 104, it may be saved to file server 114. In one embodiment, file server 114 may include a data management system that is designed to store and manage images of patient 74.

Workstations 106, 108 are coupled to hospital network 101 and, in the embodiment shown in FIG. 2, are positioned in the physician's office and the hospital administration office, respectively. Additional workstations may also be provided as part of hospital network 101 such as, for example, a workstation positioned in surgery, intensive care, etc.

Coupling electrophysiology system 50 directly to network 100 provides a number of advantages. For example, information pertaining to patient 74 may be transmitted over network 100 and stored as part of a data record for patient 74. In this configuration, users from remote locations on the network are able to access and manipulate the information acquired using system 50. For example, in one embodiment, users on the network may be able to join an EP study as it is happening. Thus, multiple users can see the results of the EP study without being in the room where it is happening. Also, a camera (e.g., video camera, periodic still camera, etc.) and a microphone may be used to acquire video and audio signals and transmit those to the users over network 100. Thus, the users on network 100 are able to observe more closely and feel more involved in the procedure without being an extra body in operating room.

In addition, system 50 may use network 100 to receive images which were acquired in the radiology laboratory and saved to file server 114. Thus, the attending physician in the EP laboratory may have quick and easy access to multiple images of heart 72 of patient 74 by way of network 100.

The images acquired over network 100 may be used to create a number of reports. For example, a report may be created at the end of each EP study summarizing the information acquired during the study, the treatment methods (e.g., RF ablation, medication, etc.), etc. The report may also include electrical and structural maps of heart 72 along with images of heart 72. By including detailed electrical information about heart 72, such as ECG readings during pacing, and a structural map or image that is correlated, and in some instances registered with the electrical information, the attending physician has a simple and easy reference for the future. It may be useful for the physician to refer to reports for research purposes or, if the problem occurs again, to determine likely causes for the recurrence.

In one embodiment, the reports may comprise vitals information as well as additional information such as the name of the physician performing the EP procedure, the name of the nurse that is present, medications patient 74 may be taking, allergies, patient history, and/or a description of the procedure. The description of the procedure may provide information about probe 56 (e.g., type of probe, location where probe 56 is inserted into the body, etc.). The reports may also include electrical information pertaining to heart 72. For example, the reports may include information resulting from pacing heart 72 (e.g., site where pacing was induced, etc.) and/or information about any induced arrhythmias and, in particular, ventricular tachycardia. The reports may also include information pertaining to a structural map of heart 72 such as a structural map of heart 72 or information pertaining to the location of probe 56 as it is moved around inside heart 72. The reports may also include information pertaining to treatments performed during the procedure. For example, the reports may include information about the location and time of an ablation. All of this information may be provided to the physician in an easy to read and understand manner. The reports may be especially useful later when examining the patient's 74 medical history to determine any problems or history of illness associated with patient 74. In addition, the reports may include one or more detailed images of heart 72 of patient 74. Thus, when reviewing the report, the physician may be able to easily refer to the unique anatomy of heart 72 off patient 74.

The following is one embodiment of a report that may be generated using system 50. The information provided in following report is only meant to show various types of information that may be provided in a report and is not meant to represent actual data obtained from a patient.
Referring Physician: Referring Physician, MD
Primary Care Physician: Attending Physician, MD
Nurse: Attending Nurse
Tech: Technician
Current Medications: None
Allergies: None
History: 40 year old male with Hepatitis C and ex-IV drug abuse with known WPW since age 17. He has had infrequent palpitations in the past but recently had an episode of prolonged palpitations and was evaluated for ablation.
   Procedure:

After informed written consent was obtained the patient was transported to the electrophysiology laboratory in the post absorptive, non-sedated state. The patient was prepped and draped in the usual sterile manner. A 1% Lidocaine solution was used for local anesthesia. A combination of Fentanyl, Droperidol and Morphine were used for conscious sedation throughout the procedure. The patient was continuously monitored throughout the case per hospital standards. The following sheaths were placed, after local anesthesia, using the Seldinger technique. In addition the following electrode catheters were placed under fluoroscopic guidance.

| | | Cath- | | | Cath- | |
|---|---|---|---|---|---|---|
| Site | Sheath | eter | Location | Location | eter | Location |
| 1 | 5 F Cordis | | HRA | HRA | | HRA |
| 2 | 6 F Cordis | | RVA | RVA | | RVA |
| 3 | 7 F Cordis | | RVOT | RVOT | | RVOT |
| 4 | 8 F Cordis | | CS | CS | | CS |
| 5 | 6.5 F Locking | | RA | RA | | RA |
| 6 | 10 F Duo | | Tricupid | Tricupid | | Tricupid |
| | | | Ann | Ann | | Ann |
| 7 | 11 F Duo | | LA | LA | | LA |
| 8 | 11 F Trio | | LV | LV | | LV |

After baseline conduction intervals were recorded, programmed extra-stimulation was performed. Atrial overdrive pacing and extra-stimulation was performed from the HRA. Ventricular overdrive pacing and extra-stimulation was performed with up to three extra-stimuli from the LV. Following intravenous administration of Procainimide programmed stimulation was repeated.

At the end of the procedure the catheters and sheaths were removed and hemostasis was achieved with pressure. The patient was transported back to the recovery room in good condition.

Results:

At baseline the patient was in atrial fibrillation with a CL of ______.
Baseline conduction intervals were:
PR ______ QRS ______ QT _____ AH _____ HV ______
Ventricular Pre-excitation was present.
Corrected sinus node recovery time was ______.
AV Nodal Conduction
   AV Nodal block cycle length _____.
   AV Nodal VA block cycle length ______.

VA conduction was not Decremental.

| Pacing Site | Refractory Site | Drive | ERP |
|---|---|---|---|
| HRA | Atrium | | |
| RVA | AV Node | | |
| RVOT | Ventricle | | |
| CS | Atrium | | |
| LA | AV Node | | |
| LV | Ventricle | | |

Results Post Procainamide Infusion:
The rhythm was SVT with a cycle length of _____.
Conduction intervals were: PR ______ QRS ______ QT ______ AH ______ HV ______
Ventricular Pre-excitation was not present.
Corrected sinus node recovery time was ______.
AV Nodal Conduction
   AV Nodal block cycle length ______.
   AV Nodal VA block cycle length ______.

VA conduction was Decremental.

| Pacing Site | Refractory Site | Drive | ERP |
|---|---|---|---|
| HRA | Atrium | | |
| RVA | AV Node | | |
| RVOT | Ventricle | | |
| CS | Atrium | | |
| LA | AV Node | | |
| LV | Ventricle | | |

Induced Arrhythmias

SVT: [symbol] Yes □ No

| Type | Induction | CL | Sustained |
|---|---|---|---|
| AVRT | | | yes |

VT: [symbol] Yes □ No

| Induction | CL | Morphology | Sustained | Termination |
|---|---|---|---|---|
| | | Right Bundle Superior Axis | yes | spontanteous |
| | | Right Bundle Inferior Axis | no | burst pacing |
| | | Left Bundle Superior Axis | yes | cardioversion |
| | | Left Bundle Inferior Axis | no | medication |

Mapping:

After the baseline study was completed extensive endocardial mapping was performed.

[Image of heart 72 shown here (e.g., CT image, MR image, ultrasound image, PET image, etc.]

Ablation:

| | | | | | | |
|---|---|---|---|---|---|---|
| Number | Time | Location | Target Arrhythmia | Catheter | NIBP | Pulse |

Post Ablation Extra-Stimulation:
The rhythm was sinus rhythm with a cycle length of ______.
Conduction intervals were: PR ______ QRS ______ QT ______ AH ______ HV ______
Ventricular Pre-excitation was present.
Corrected sinus node recovery time was ______.
AV Nodal Conduction
   AV Nodal block cycle length ______.
   AV Nodal VA block cycle length ______.

VA conduction was not Decremental.

| Pacing Site | Refractory Site | Drive | ERP |
|---|---|---|---|
| HRA | Atrium | | |
| RVA | AV Node | | |
| RVOT | Ventricle | | |
| CS | Atrium | | |
| LA | AV Node | | |
| LV | Ventricle | | |

Induced Arrhythmias

SVT: [symbol] Yes □ No

| Type | Induction | CL | Sustained |
|---|---|---|---|
| A tach | | | yes |

Vitals Monitoring:

| | | | | | | |
|---|---|---|---|---|---|---|
| Time | NIBP | Pulse | Resp | Temp | SpO₂ | EtCO₂ |

Findings:
1.
2.
3.
   Plan:
2.
3.
______, M.D.
Director of Electrophysiology Laboratory

The report shown above is only one example of a suitable report. Accordingly, numerous alterations may be made to the format of the information and what information is included.

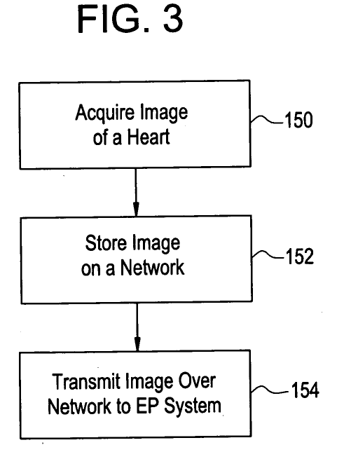

Referring to FIG. 3, a method is shown for providing an image over network 100 to system 50. Before patient 74 enters the EP laboratory, patient 74 is sent to the radiology laboratory to acquire an image of heart 72 of patient 74. As mentioned previously, the image may be any suitable image that is helpful to refer to in performing an EP procedure. At step 150, the image of heart 72 is acquired. The image may be acquired using any of a number of imaging modalities. Typically, the image is acquired using a CT, MR, ultrasound, or PET imaging system. However, other imaging systems may be used as would be recognized by one of ordinary skill in the art. Although step 150 refers to acquiring an image, it should be understood that many of the systems used to acquire an image output image data which is used to construct an image of heart 72. Reference to acquiring an image is meant to include acquiring image data that is later used to construct an image as well as acquiring an actual image. In many situations, the image data includes a plurality of image slices each of which represents a cross sectional slice of heart 72.

At step 152, the image is stored on network 100. Typically, this is done by storing the image directly to file server 114 where the image is now available to a wide variety of workstations on network 100. As mentioned previously, the image may be stored directly to file server 100 from the imaging system used to acquire the image or from workstation 104 which is located in or near the radiology laboratory. Also, the image may be stored in a database which is used to organize and manage a large number of images.

At step 154, the image is transmitted over network 100 directly to electrophysiology system 50. In practice, the image is received by system 50 when the user of system 50 pulls it off of file server 114. Once the image is received by system 50, it may be used as a reference for the physician during the EP procedure.

In addition to receiving the image, system 50 may comprise image processing tools which are used to manipulate the image. In one embodiment, the image may be manipulated by processing the image data which is used to construct the image in a variety of ways. Also, the image processing tools included as part of system 50 may be used in connection with images that are constructed based on a plurality of image slices each of which represent a slice of heart 72 and other images not made up of image slices. Many of the image processing tools described herein are particularly applicable to manipulating CT images. However, the tools may be used to manipulate other images and should not be limited to only manipulating CT images.

In one embodiment, the image processing tools included with system 50 may comprise a virtual endoscope tool. This tool is used to view the interior of closed cavities or otherwise opaque lumens inside the body of patient 74. This tool provides a three dimensional view of the cavity or lumen as though it were empty and expanded. In other words, the material inside the cavity or lumen (e.g., blood) is not visible in the image but the interior side walls of the cavity or lumen are visible. Using this tool, the user can travel or “fly” through the cavity or lumen observing the interior side walls. This tool may be useful in planning an EP procedure by allowing the physician to visualize the route probe 56 will take as it travels through the vascular system of patient 74 and into heart 72.

In another embodiment, the image processing tools may include a volume rendering tool. In general, the volume rendering tool allows the image data (e.g., CT or MR image slices) to be manipulated to provide a detailed three dimensional image. For example, a CT image of heart 72 may be volume rendered to produce a three dimensional image of the heart. The volume rendered image enables the user to perform additional analysis such as further segmentation, region of interest (ROI) isolation, bone removal, and measurements. Also, the user may be able to manipulate color, opacity, brightness, and to sub-select features of heart 72 that are of interest. In a further embodiment, the volume rendering tool may be configured to automatically build the image from the image slices simultaneously with standard reconstruction. This may be fully automatic, interactive with limited user interaction, or manual with complete user control.

In another embodiment, the image processing tools may include an image segmentation tool. The image segmentation tool is used to isolate areas of interest in the image from other portions of the image. For example, if the user desires to view only heart 72 and the coronary vessels surrounding the heart 72, then the image segmentation tool may be used to remove any portion of the image that shows other features or structures. In one embodiment, the image segmentation tool may be used to create a three dimensional image of selected features or structures inside the body.

In another embodiment, the image processing tools may include heart specific three dimensional volume rendering tools. These tools may be used to automatically isolate the heart in an image (e.g., CT image) and create a three dimensional view. Protocols may be included which optimize the three dimensional image to show different structures such as coronary arteries, calcified plaque, heart chambers, bypass grafts, stents, etc.

In another embodiment, the image processing tools may include simulation tools. The simulation tools may include automatic outlining of features in the image. The automatic outlining tool may include erosion and dilation functions and be capable of breaking small bridges between different volumes before contouring the image. Another simulation tool that may be included is contour tracing. The contour tracing tool allows the user to trace the external surface of a structure on parallel planes using a cursor, then define the complete volume by interpolation. A magnetic cursor may be used to follow structure boundaries. Another simulation tool that may be included is contour editing, which is used to edit the shape of a contour using the cursor. Yet another simulation tool that may be included is volume expansion. The volume expansion tool is used to expand structures to create a new volume with, in one embodiment, user defined margins in the anterior, posterior, inferior, superior, left, and right directions. Yet another simulation tool that may be included is the conform tool. The conform tool is used to conform the collimator or a block around two or more defined structures with a specified margin. The simulation tools may provide a variety of views such as axial, sagittal, coronal, oblique, or three dimensional.

In another embodiment, the image processing tools may include cardiac imaging tools. When an image is taken of a heart 72 using an imaging system that acquires a plurality of image slices, it is desirable to gate the image acquisition to coincide with a certain point on the cardiac cycle so that heart 72 is in the same position each time an image slice is acquired. In some situations, it may be desirable to gate the acquisition of the image slices to more than one point in the cardiac cycle. For example, it is often desirable to have multiple images of heart 72 at various points in the cardiac cycle to assist in further diagnosis and treatment of patient 74. The cardiac imaging tools may be used to view and manipulate the multiple images acquired in this manner.

In another embodiment, the image processing tools may include cardiac scoring tools, which are used to provide coronary artery calcification scores. In general, images that are used for calcification scoring are acquired using one of two EKG gating methods: prospective gating and retrospective gating. Scoring of the images or ROIs in the images is typically divided into three steps: extracting intensity histograms for each image/region, computing image/region scores based on particular scoring procedures, and combining region scores for individual images into composite scores using slice-thickness weight. The calcium score may be shown as a total calcium score, individual coronary artery score, or individual slice calcium score.

In yet another embodiment, the image processing tools may include cardiac functional analysis tools, which are used to semi-automatically or manually calculate left ventricular and right ventricular functional parameters of heart 72. Typically, the cardiac functional analysis tools are used in conjunction with a CT image. The cardiac functional analysis tools may be used to create ventricular volume graphs for one or both the left and right ventricles. Also, the cardiac functional analysis tools may be used to create a wall motion graph showing the amount of motion of the wall of heart 72. Further, the tools may be used to create a bullseye plot of the thickness of heart 72 as well as a three dimensional contour animation of heart 72.

In yet another embodiment, the image processing tools may include coronary vessel analysis tools. These tools may be used to provide auto vessel tracking by depositing a single seed in distal vessel. Also, these tools may be used to display vessels in curved reformat, oblique, and lumen view as well as provide automatic centerline detection of a vessel. In addition, these tools may be used to measure the vessel diameter and size as well as provide stenosis sizing. In a further embodiment of the vessel analysis tools, they may be used to provide maximum, minimum, and mean intraluminal diameter measurements and cross-sectional areas of true orthogonal sections of the aortoiliac systems at selected anatomical points.

In yet another embodiment, the image processing tools include a coronary vessel tree tool. This tool may be used to automatically isolate the heart chambers and surrounding vessels and show them in a volume rendered or MIP projection. The image may be reformed in the coronal or sagittal plane.

The image processing tools provided as part of system 50 allow the physician performing the EP procedure to modify and reconstruct the images as desired. This allows the physician greater flexibility in the use of these images.

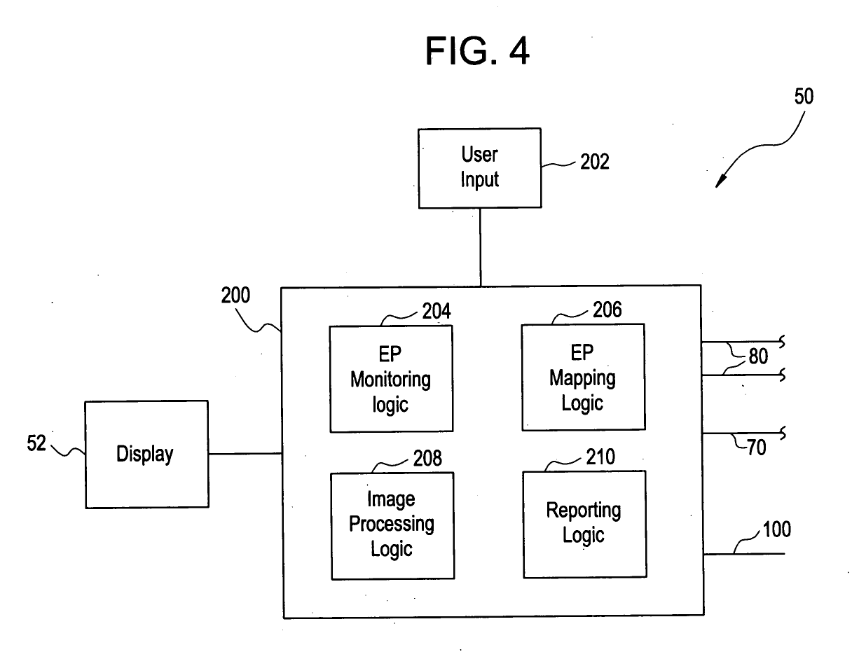

Referring to FIG. 4, a block diagram shows the logic that may be included as part of system 50 according to another embodiment. In this embodiment, system 50 is shown comprising logic processes 200, display 52, user input device 202, input receivers 80, probe body 70, and a connection to network 100. Display 52, input receivers 80, and probe body 70 may be configured as described previously. Input device 202, also described generically in reference to FIG. 1, is used to provide input in the form of commands, etc. to system 50.

Logic processes 200 comprise EP monitoring logic 204, EP mapping logic 206, image processing logic 208, and reporting logic 210. All of the various logic is in the form of computer readable instructions on system 50. EP monitoring logic 204 and EP mapping logic 206 generally correspond to the logic used to operate an EP monitoring system and an EP mapping system, respectively. Accordingly, EP mapping logic 206 comprises logic which is used to determine the position of probe 56. EP mapping logic 206 may also include logic which is used to create a structural map of heart 72 using multiple positions of probe 56. EP monitoring logic 204 may be used to acquire detailed electrical information pertaining to heart 72. For example, EP monitoring logic 204 may be used to pace heart 72 and record or log the electrical information pertaining to heart 72 as it is paced.

Reporting logic 210 may be used to generate a report using the information acquired by system 50 during the EP study. The report may be generated by populating fields in a template report using data stored in a database in system 50 or on network 100. As mentioned previously, the report may include an image (e.g., three dimensional image) acquired using a CT, MR, ultrasound, or PET imaging system.

Image processing logic 208 includes one or more of the image processing tools described above. Thus, image processing logic is used to manipulate the images acquired by system 50 over network 100. Logic processes 200 are generally instructions which are executable by data processing system 54.

The construction and arrangement of the elements described herein are illustrative only. Although only a few embodiments have been described in detail in this disclosure, those of ordinary skill who review this disclosure will readily appreciate that many modifications are possible without departing from the spirit of the subject matter disclosed herein. Accordingly, all such modifications are intended to be included within the scope of the methods and systems described herein. The order or sequence of any process or method steps may be varied or re-sequenced according to alternative embodiments. Other substitutions, modifications, changes and omissions may be made in the design, operating conditions and arrangement of the embodiments without departing from the spirit and scope of the methods and systems described herein.

1. A system comprising:
one or more probes configured to be positioned inside a heart of a patient;
a processor communicatively coupled to the one or more probes, the processor being used to process electrical information pertaining to the heart, the electrical information being sensed using the one or more probes;
a display communicatively coupled to the processor, the display being used to display an image of the heart;
image processing tools which are used by the processor to manipulate the image.

2. The system of claim 1, wherein the image is acquired using a computed tomography imaging system, a magnetic resonance imaging system, an ultrasound imaging system and/or a positron emission tomography imaging system.

3. The system of claim 1, wherein the processor is used to process position information which pertains to the position of at least one of the one or more probes positioned in the heart.

4. The system of claim 3, wherein the position information is used to create a structural map of the heart.

5. The system of claim 1, wherein the image processing tools include at least one of a volume rendering tool, a virtual endoscope tool, a coronary vessel analysis tool, an image reconstruction tool, and an image segmentation tool.

6. The system of claim 1, wherein the image is acquired prior to the probe being positioned in the heart.

7. The system of claim 1, wherein the image is constructed based on a plurality of image slices each of which represents a cross sectional slice of the heart, and wherein the image processing tools are used to manipulate the image by manipulating the plurality of image slices.

8. A computer based system comprising:
electrophysiology monitoring logic which is used to monitor and control one or more probes positioned inside a heart; the one or more probes being used to sense electrical information pertaining to the heart;
mapping logic which is used create a structural map of the heart by determining the position of at least one of the one or more probes inside the heart; and
image processing logic which is used to manipulate an image of the heart.

9. The system of claim 8, wherein the image is a computed tomography image, magnetic resonance image, ultrasound image and/or positron emission tomography image.

10. The system of claim 8, wherein the image processing logic includes at least one of the following types of logic: volume rendering logic, virtual endoscope logic, image reconstruction logic, and image segmentation logic.

11. The system of claim 8, further comprising reporting logic which is used to create a report which includes the electrical information and the image.

12. The system of claim 8, wherein the electrophysiology monitoring logic comprises pacing logic which is used to pace the heart.

13. The system of claim 8, wherein the image is a three dimensional image.

14. A system comprising:
one or more probes configured to be positioned inside a heart, at least one of the one or more probes being used to sense electrical information pertaining to the heart;
a data processing system communicatively coupled together and communicatively coupled to the one or more probes, the data processing system being configured to store position information pertaining to a position of at least one of the one or more probes, the data processing system also being configured to store an image of the heart and image processing tools;
wherein the image processing tools are used to manipulate the image.

15. The system of claim 14, wherein the image is acquired using a computed tomography imaging system, a magnetic resonance imaging system, an ultrasound imaging system and/or a positron emission tomography imaging system.

16. The system of claim 14, wherein the data processing system uses the position information to create a structural map of the heart.

17. The system of claim 14, wherein the image processing tools include at least one of a volume rendering tool, a virtual endoscope tool, an image reconstruction tool, and an image segmentation tool.

18. The system of claim 14, wherein the image is a three dimensional image.

19. A combination system comprising:
an electrophysiology monitoring system which is configured to be communicatively coupled to one or more probes positioned inside a heart, the one or more probes being configured to sense electrical information pertaining to the heart;
an electrophysiology three-dimensional mapping system which is configured to receive position information pertaining to the position of the one or more probes, the position information being used to create a three-dimensional structural map of the heart, the electrophysiology monitoring system and the electrophysiology three-dimensional mapping system being communicatively coupled together;
image processing logic which is used to manipulate an image of the heart.

20. The system of claim 19, wherein the image processing logic includes at least one of the following types of logic: volume rendering logic, virtual endoscope logic, image reconstruction logic, and an image segmentation tool.

21. The system of claim 19, wherein the image is a computed tomography image, magnetic resonance image, ultrasound image and/or positron emission tomography image.

22. The system of claim 19, wherein the combination system is configured to generate a report which includes the image.

23. A system comprising:
one or more probes configured to be positioned inside a heart of a patient;
a processor communicatively coupled to the one or more probes, the processor being used to process electrical information pertaining to the heart, the electrical information being sensed using the one or more probes;
image processing tools in the form of computer readable instructions, the image processing tools being used to manipulate an image of a heart, the image being constructed based on a plurality of image slices each of which represents a cross sectional slice of the heart;
a user interface communicatively coupled to the processor, the user interface being configured to display the image.
